(19) United States
(12) Patent Application Publication
(10) Pub. No.: US 2025/0169991 A1
(43) Pub. Date: May 29, 2025

(54) Auto Darkening Filter Lens and Control Panel for Welding Helmet

(71) Applicant: The ESAB Group Inc., North Bethesda, MD (US)

(72) Inventors: Kevin Beckerdite, Azle, TX (US); Muhammad Khan, Prosper, TX (US); Azfar Kamal, Frisco, TX (US); Matthew Tanner, Whitesboro, TX (US)

(21) Appl. No.: 19/038,895

(22) Filed: Jan. 28, 2025

Related U.S. Application Data
(63) Continuation of application No. PCT/US2023/071308, filed on Jul. 31, 2023.
(60) Provisional application No. 63/394,011, filed on Aug. 1, 2022.

(51) Int. Cl. A61F 9/06

(52) U.S. Cl. CPC A61F 9/061

(57) ABSTRACT

A control panel for an ADF including a series of buttons or actuators arranged in a grid-like array having two rows and three columns and a series of LEDs vertically aligned with one another and located adjacent to the array of buttons. The LEDs may be more centrally located between the first and second sides of the ADF, while the array of buttons may be located more proximate to the second side of the ADF. The control panel may be disposed more proximate to the bottom side of the ADF than the top side of the ADF. Each of the buttons of the array of buttons may extend or protrude from the surface of the control panel. The spacing, arrangement, size, and protruding nature of each of the buttons enables a user wearing welding gloves to easily locate and operate the control panel of the ADF.

### CROSS-REFERENCE TO RELATED APPLICATIONS

This patent application is a continuation of, and claims priority to and the benefit of, International Patent Application No. PCT/US2023/071308, filed Jul. 31, 2023, entitled “AUTO DARKENING FILTER LENS AND CONTROL PANEL FOR WELDING HELMET,” which claims priority to and the benefit of U.S. Provisional Patent Application No. 63/394,011, filed Aug. 1, 2022, entitled “AUTO DARKENING FILTER LENS CONTROL PANEL”. The entire disclosures of both of the above-identified patent applications are incorporated herein by reference in their entireties.

### TECHNICAL FIELD

The present disclosure is directed toward a welding helmet and, in particular, to a control panel of an auto darkening filter lens of a welding helmet.

### SUMMARY OF THE INVENTION

The present invention is directed to a control panel for an auto darkening filter lens or auto darkening filter (“ADF”). In one embodiment, the control panel may include a series of buttons or actuators arranged in a grid-like array. In one example embodiment, the array has two rows and three columns. Each of the buttons may be equally spaced from the other adjacent buttons in both lateral (e.g., horizontal) and longitudinal (e.g., vertical) directions. In addition, the buttons of the array may be equal in shape and size to one another and may extend or protrude from a surface or face of the control panel.

The control panel may further include a series of light emitting diodes (“LEDs”) that are vertically aligned with one another and located adjacent to the array of buttons. In some embodiments, the LEDs may be more centrally located on the ADF between the first and second sides, while the array of buttons may be located more proximate to the second side than the first side of the ADF. The control panel containing the array of buttons and the LEDs may be disposed more proximate to the bottom side of the ADF (i.e., below a viewing window) than the top side of the ADF. The spacing, arrangement, size, and protruding nature of each of the buttons of the array of buttons enables a user wearing welding gloves to easily locate and operate the buttons of the control panel of the ADF.

In one example embodiment of the invention, an auto darkening filter lens of a welding helmet, comprises a body including: a viewing window through which a user wearing the welding helmet can view outside of the welding helmet; and a control panel, the control panel including: a surface defining a plurality of openings formed therein; a plurality of actuators, at least one of the plurality of actuators being related to an operation feature of the auto darkening filter lens, and each of the plurality of actuators being located in one of the plurality of openings in the surface; and a plurality of light emitting diodes (LEDs), wherein each of the plurality of LEDs relates to an operation of the auto darkening filter lens.

One aspect of the auto darkening filter lens is that the plurality of actuators is arranged in an array of rows and columns, and each of the plurality of actuators is spaced apart from another one of the plurality of actuators by a distance. Another aspect is that the array of rows and columns includes two rows and three columns. Another aspect is that the body includes a top end, a bottom end opposite to the top end, a first side, and a second side opposite to the first side, and the plurality of actuators are closer to the first side than to the second side, and are closer to the bottom end than to the top end. Yet another aspect is that the control panel includes a screen that is closer to the second side than to the first side, and closer to the bottom end than to the top end.

Another aspect is that each of the plurality of actuators is a button having a width of at least 10.0 mm and a height of at least 9.0 mm. Another aspect is that each of the plurality of actuators has a width of at least 12.0 mm and a height of at least 10.0 mm. Yet another aspect is that each of the plurality of actuators is spaced apart from another one of the plurality of actuators in a horizontal direction by a distance greater than 3.0 mm, and is spaced apart from another one of the plurality of actuators in the horizontal direction by a distance greater than 3.0 mm. An alternative aspect is that each of the plurality of actuators is spaced apart from another one of the plurality of actuators in the horizontal direction by a distance greater than 4.0 mm, and is spaced apart from another one of the plurality of actuators in the horizontal direction by a distance greater than 4.0 mm. Yet another alternative aspect is that each of the plurality of actuators has a substantially rectangular shape.

In another example embodiment of the invention, a control panel for an auto darkening filter lens of a welding helmet, comprises a surface defining a plurality of openings formed therein; a plurality of actuators, at least one of the plurality of actuators being related to an operation feature of the auto darkening filter lens, and each of the plurality of actuators being located in one of the plurality of openings in the surface; and a plurality of light emitting diodes (LEDs), wherein each of the plurality of LEDs relates to an operation of the auto darkening filter lens.

One aspect of the control panel is that the plurality of actuators is arranged in an array of rows and columns, and each of the plurality of actuators is spaced apart from another one of the plurality of actuators by a distance. Another aspect is that the control panel includes a top end, a bottom end opposite to the top end, a first side, and a second side opposite to the first side, and the plurality of actuators are closer to the first side than to the second side. Yet another aspect is that the control panel includes a screen that is closer to the second side than to the first side. In another aspect, each of the plurality of actuators is a substantially rectangular button having a width of at least 10.0 mm and a height of at least 9.0 mm, and each of the plurality of actuators is spaced apart from another one of the plurality of actuators in a horizontal direction by a distance greater than 3.0 mm, and is spaced apart from another one of the plurality of actuators in the horizontal direction by a distance greater than 3.0 mm.

In another example embodiment, a welding helmet comprises a shell; a headgear coupled to the shell, the headgear being engageable with a user's head; and an auto darkening filter lens coupled to the shell, the auto darkening filter lens including: a body having a viewing window and a control panel, the control panel including: a surface defining a plurality of openings formed therein; a plurality of actuators, at least one of the plurality of actuators being related to an operation feature of the auto darkening filter lens, each of the plurality of actuators being located in one of the plurality of openings in the surface; and a plurality of light emitting diodes (LEDs), wherein each of the plurality of LEDs relates to an operation of the auto darkening filter lens.

One aspect of the welding helmet is that the plurality of actuators is arranged in an array of two rows and three columns, and each of the plurality of actuators is spaced apart from another one of the plurality of actuators by a distance. Another aspect is that the body of the auto darkening filter lens includes a top end, a bottom end opposite to the top end, a first side, and a second side opposite to the first side, and the plurality of actuators are closer to the first side than to the second side, and are closer to the bottom end than to the top end. Yet another aspect is that the control panel includes a screen that is closer to the second side than to the first side, and closer to the bottom end than to the top end. Another aspect is that each of the plurality of actuators is a substantially rectangular button having a width of at least 10.0 mm and a height of at least 9.0 mm, and each of the plurality of actuators is spaced apart from another one of the plurality of actuators in a horizontal direction by a distance greater than 3.0 mm, and is spaced apart from another one of the plurality of actuators in the horizontal direction by a distance greater than 3.0 mm.

Other systems, methods, features and advantages will be, or will become, apparent to one with skill in the art upon examination of the following figures and detailed description. All such additional systems, methods, features and advantages are included within this description, are within the scope of the claimed subject matter.

### BRIEF DESCRIPTION OF THE DRAWINGS

The helmet, ADF, headgear, buttons, LEDs, apparatuses, and other components presented herein may be better understood with reference to the following drawings and description. It should be understood that some elements in the figures may not necessarily be to scale and that emphasis has been placed upon illustrating the principles disclosed herein. In the figures, like-referenced numerals designate corresponding parts/steps throughout the different views.

FIG. 1 illustrates a front perspective view of a welding helmet equipped with an ADF, according to an example embodiment of the present disclosure.

FIG. 2 illustrates a rear perspective view of the welding helmet illustrated in FIG. 1.

FIG. 3 illustrates a front perspective view of the welding helmet illustrated in FIG. 1 with the shield lens removed.

FIG. 4 illustrates a rear perspective view of the welding helmet illustrated in FIG. 1 with the headgear removed.

FIG. 5 illustrates an exploded view of the welding helmet illustrated in FIG. 1.

FIG. 6 illustrates rear perspective view of the ADF equipped on the welding helmet illustrated in FIGS. 1 and 2.

FIG. 7 illustrates a front perspective view of the ADF illustrated in FIG. 6.

FIG. 8 illustrates a rear view of the ADF illustrated in FIG. 6.

FIG. 9 illustrates and isolated elevational view of the control panel of the ADF illustrated in FIG. 6.

FIG. 10 illustrates a schematic view of a portion of the control panel illustrated in FIG. 9.

FIG. 11 illustrates a bottom view of the ADF illustrated in FIG. 6.

FIG. 12 illustrates a schematic diagram of a few components of an ADF, according to an example embodiment of the present disclosure.

### DETAILED DESCRIPTION

In the following detailed description, reference is made to the accompanying figures which form a part hereof wherein like numerals designate like parts throughout, and in which is shown, by way of illustration, embodiments that may be practiced. It is to be understood that other embodiments may be utilized, and structural or logical changes may be made without departing from the scope of the present disclosure. Therefore, the following detailed description is not to be taken in a limiting sense, and the scope of embodiments is defined by the appended claims and their equivalents.

Aspects of the disclosure are disclosed in the description herein. Alternate embodiments of the present disclosure and their equivalents may be devised without parting from the spirit or scope of the present disclosure. It should be noted that any discussion herein regarding “one embodiment,” “an embodiment,” “an exemplary embodiment,” and the like indicate that the embodiment described may include a particular feature, structure, or characteristic, and that such particular feature, structure, or characteristic may not necessarily be included in every embodiment. In addition, references to the foregoing do not necessarily comprise a reference to the same embodiment. Finally, irrespective of whether it is explicitly described, one of ordinary skill in the art would readily appreciate that each of the particular features, structures, or characteristics of the given embodiments may be utilized in connection or combination with those of any other embodiment discussed herein.

For the purposes of the present disclosure, the phrase “A and/or B” means (A), (B), or (A and B). For the purposes of the present disclosure, the phrase “A, B, and/or C” means (A), (B), (C), (A and B), (A and C), (B and C), or (A, B and C). The terms “comprising,” “including,” “having,” and the like, as used with respect to embodiments of the present disclosure, are synonymous.

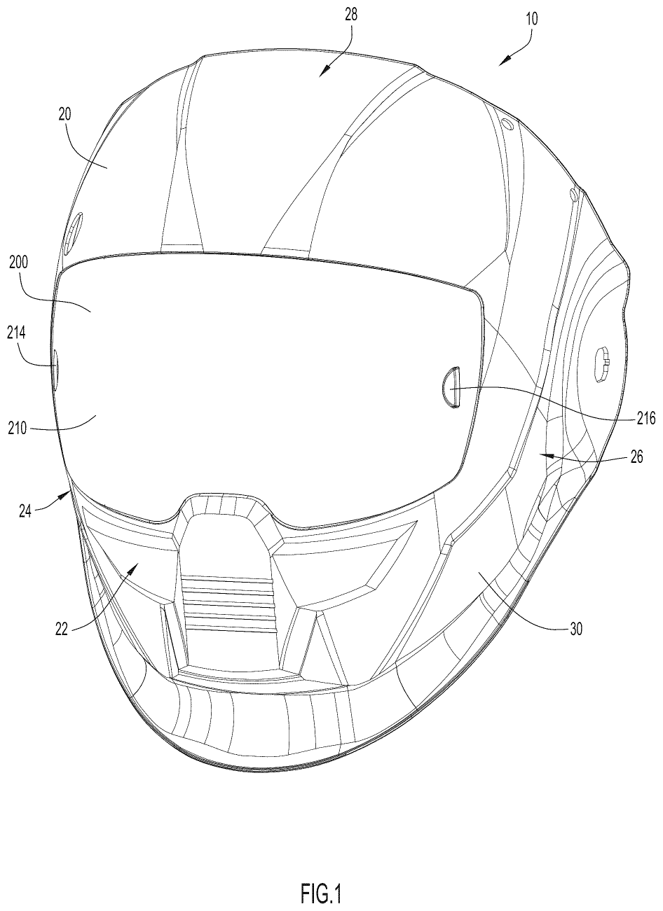

Turning to FIGS. 1-4, an exemplary embodiment of a welding helmet is illustrated. The welding helmet 10 may include, among other components, a shell 20, a headgear unit 100, a shield lens 200, and an auto darkening filter lens or ADF 300. As illustrated, the welding helmet 10 may have a partial enclosure shell 20, where the shell 20 of the welding helmet 10 only partially encloses the head of the user wearing the welding helmet 10 (e.g., leaves the rear side of the user's head exposed). In other embodiments, the shell 20 of the welding helmet 10 may fully enclose the user's head or may be a face shield that is positioned in front of the user's face and does not enclose the user's head.

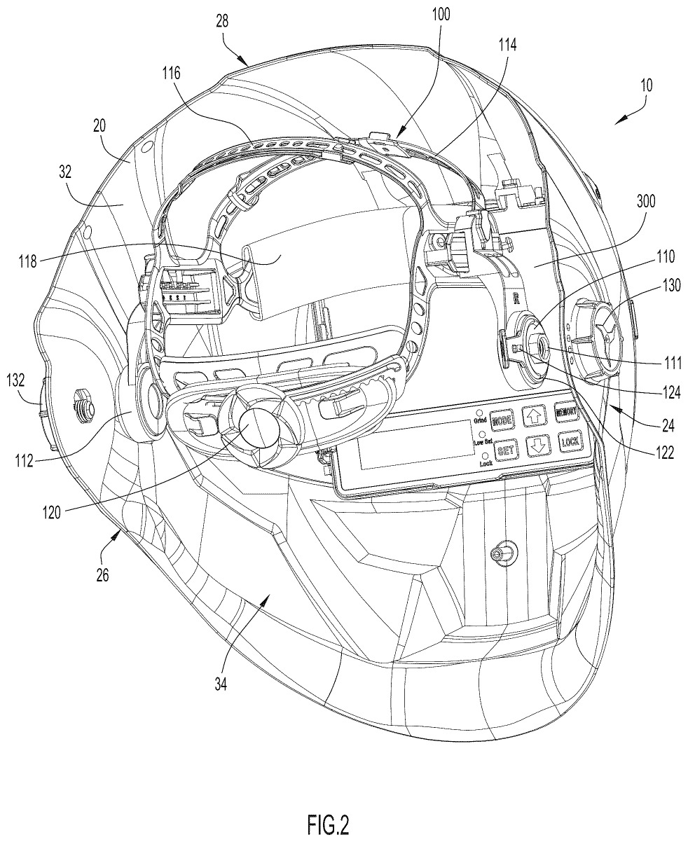

As illustrated in FIG. 1, the shell 20 may include a front side 22, a first side 24 extending rearwardly from the front side 22, a second side 26 opposite the first side 24 that also extends rearwardly from the front side 22, and a top side 28 that extends rearwardly from the front side 22 and spans between the first side 22 and the second side 24. The shell 20 may further include an exterior or outer surface 30 (see FIG. 1), and an interior or inner surface (see FIG. 2). The various sides 22, 24, 26, and 28 of the shell 20 may collectively define an interior volume or cavity 34. The cavity 34 is sized to receive at least a portion of a user's head.

Returning to FIG. 1, the welding helmet 10 includes a shield lens 200 that is removably coupled thereto. The shield lens 200 has a generally curved configuration, and includes an outer surface 210. Proximate to opposite ends of the shield lens 200 are structures used to mount the shield lens 200 to the shell 20. In this embodiment, shield lens 200 includes a notch 214 that receives a projection on the shell 20 and another notch 216 that receives another projection on the shell 20. The notches 214 and 216 and the corresponding projections facilitate mounting the shield lens 200.

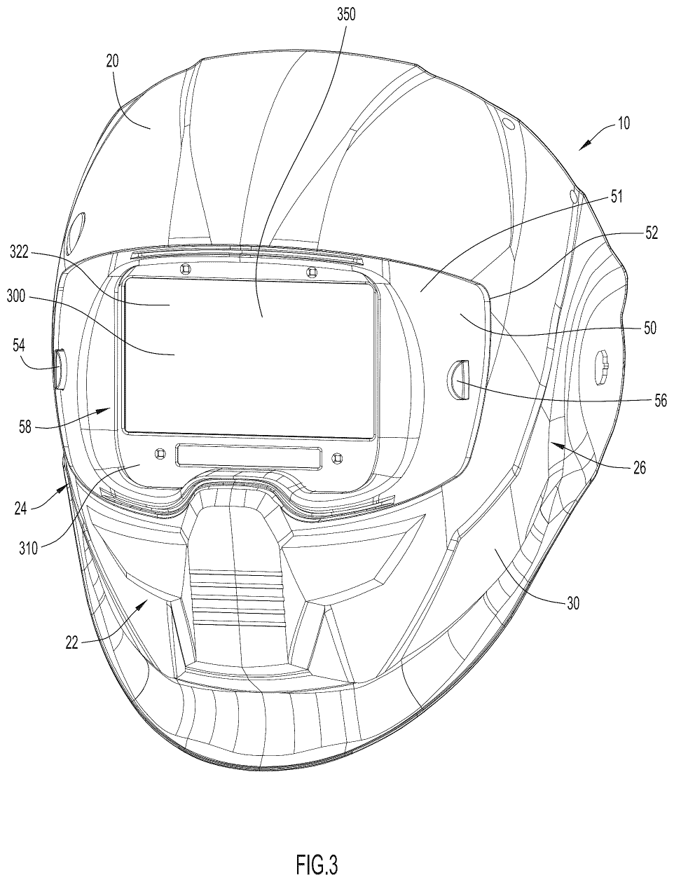

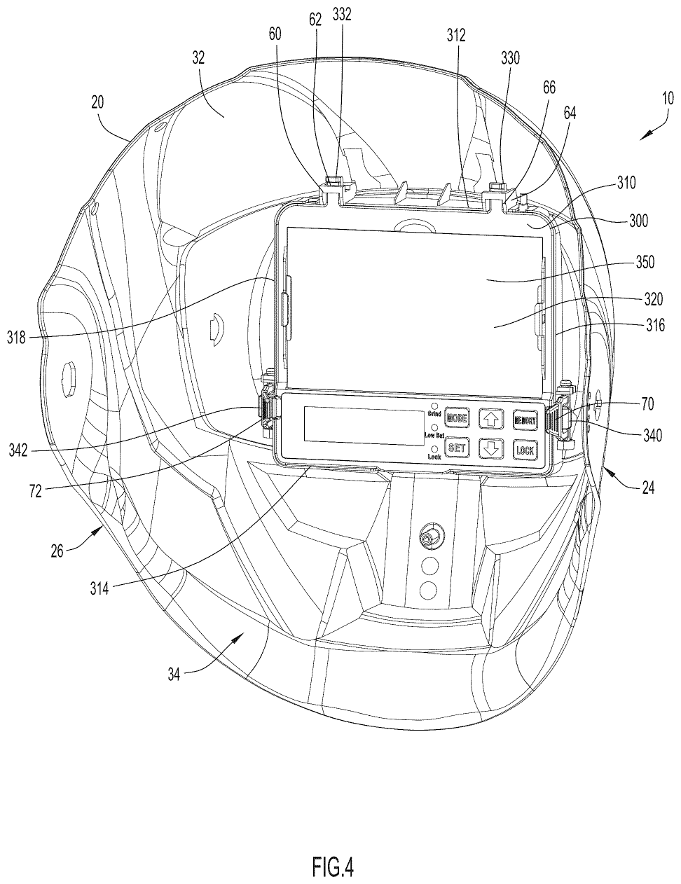

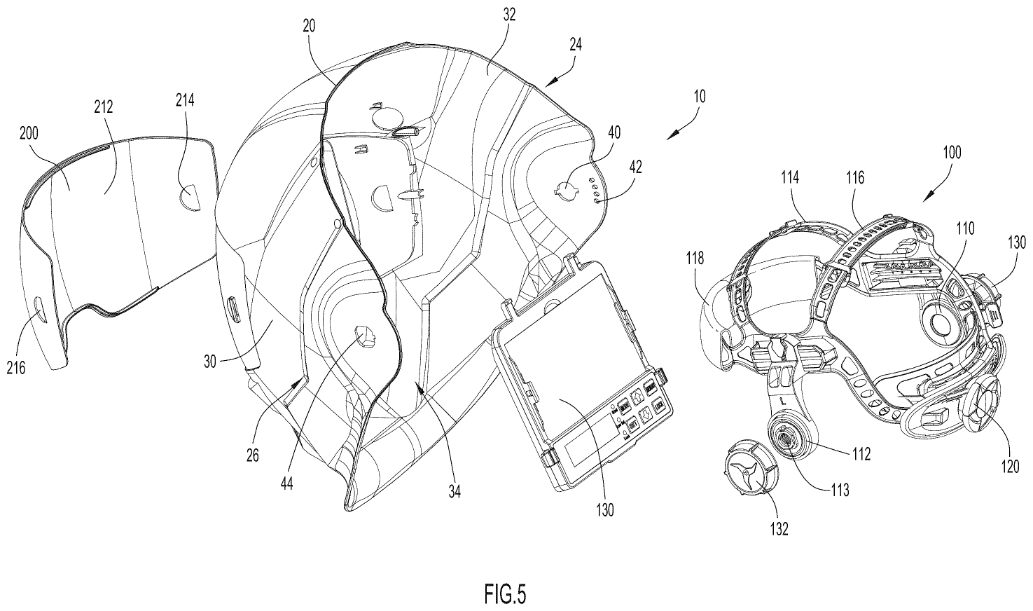

Turning to FIG. 3, the front side 22 of the shell 20 may include an opening 58 that extends through the shell 20 from the exterior surface 30 to the interior surface 32. In some embodiments, like that illustrated in FIGS. 1 and 2, the shield lens 200 may be removably attached to the exterior surface 30 of the shell 20 at the front side 22 of the shell 20 such that the shield lens 200 spans across the opening 58. As illustrated in FIGS. 4 and 5, and as further explained below, the ADF 300 may be removably coupled to the interior surface 32 of the shell 20 such that the ADF 300 is disposed proximate to the opening 58 in the shell 20 and viewable through the opening 58.

Turning to FIG. 2, the headgear 100 of the welding helmet 10 may be removably and repositionably coupled to the shell 20 of the welding helmet 10. In an example embodiment, the headgear 100 is coupled to the shell 20 via apertures or slots disposed on the first side 24 and the second side 26 of the shell 20. The headgear 100 may be configured to receive a user's head in order to position the shell 20 with respect to the user's head, and, in particular, the user's face. In addition, the headgear 100 may be configured to adjust the position of the shell 20 with respect to the headgear 100, and thus, the head of the user wearing the welding helmet 10. In other words, the headgear 10 may facilitate the tilting, rotation, and translation of the shell 20 with respect to both the headgear 100 and the head of the user wearing the welding helmet 10. The headgear 100 may also be adjustable to accommodate the various sizes and shapes of the heads of different users.

In FIG. 2, the various components of this embodiment of a headgear or headgear unit 100 are illustrated. The headgear 100 includes spaced apart support structures or bands 114 and 116 that engage the top surface of the user's head. The headgear 100 also includes a forehead portion 118 at its front end and an adjustment mechanism 120 at its rear end. The forehand portion 118 rests against the forehead of the user. The adjustment mechanism 120 includes a rotatable member, such as a dial or knob, that engages one or more gear or toothed members to allow for manipulation by a user to loosen or tighten the headgear 100 so that it fits the user's head.

The headgear 100 also includes two mounting portions located on opposite sides of the headgear 100. Mounting portion 110 is located on one side of the headgear 100 and another mounting portion 112 is located on an opposite side of the headgear 100. Each of the mounting portions 110 and 112 has an outwardly extending projection, with projection 111 on mounting portion 110 being shown in FIG. 2.

A coupler 130 is positioned proximate to the exterior surface 30 of the shell 20. The coupler 130 engages with the projection 111 of mounting portion 110 to couple the headgear 100 to the shell 20. Similarly, another coupler 132 is positioned proximate to the exterior surface 30 of the shell 20. This coupler 132 engages with the projection 113 of mounting portion 112 to couple the headgear 100 to the shell 20 as well. The mounting portions 110 and 112 and the couplers 130 and 132 are used to couple the headgear 100 to the shell 20.

In this example embodiment, mounting portion 110 includes a plate 122 extending therefrom that has a projection 124. The projection 124 engages one of several openings formed in the shell 20. When the projection 124 is engaged with an opening, the headgear 100 is secured in its position relative to the shell 20. These openings are discussed in greater detail below.

In FIG. 2, the position of the ADF 300 inside of the cavity 34 is shown. The ADF 300 is located proximate to the front of the shell 20. Referring to FIG. 3, the shield lens 200 has been removed from the shell 20. In this embodiment, the exterior surface 30 of the front side 22 includes a recess 50 defined by edge 52 and by a lower surface 51. The edge 52 matches the outer perimeter of the shield lens 200. As a result, shield lens 200 can fit into the recess 50. As mentioned above, the shield lens 200 has a pair of notches 214 and 216. The surface 51 includes a pair of projections 54 and 56 located proximate to opposite ends of the recess 50. Projection 54 engages with notch 214 in shield lens 200 and projection 56 engages with notch 216 in shield lens 200.

With the shield lens 200 removed, the ADF 300 is visible from the outside of the shell 20. The ADF 300 has a body 310 and a viewing window or viewing pane 350. In this embodiment, the body 310 surrounds the perimeter of the viewing window 350. The viewing window 350 and the body 310 collectively have an exterior or outer side 322 that faces outward.

Referring to FIG. 4, a perspective view of the interior of the shell 20 is illustrated. The headgear 100 has been decoupled from the shell 20, which allows the features of the ADF 300 to be shown. The welding helmet 10 with the shell 20 having the ADF 300 mounted thereto, as described in detail below.

As described above, the shell 20 has opposite sides 24 and 26 and an interior surface 32 defines a cavity 34. In the cavity 34, the interior surface 32 includes a projection 60 that includes a slot 62 and another projection 64 that includes a slot 66. In addition, the interior surface 32 also includes a pair of engagement mechanisms 70 and 72.

In FIG. 4, the inner side of the ADF 300 is shown. The viewing window or pane 350 has an interior or inner side 320. In this embodiment, the body 310 of the ADF 300 extends to the inner side as well. As illustrated, the ADF is a substantially rectangular prism with a body 310 having a top side or top end 312, a bottom side or bottom end 314 opposite the top end 312, a first end 316 spanning between the top end 312 and the bottom end 314, and a second end 318 opposite the first end 316 that also spans between the top end 312 and the bottom end 314.

The interior or inner side 320 of the ADF 300 spans between each of the ends 312, 314, 316, and 318. Similarly, the opposite exterior side 322 also spans between each of the ends 312, 314, 316, and 318. Extending from the top end 312 of the ADF 300 is a pair of extension members 330 and 332 that are configured to be inserted into the slots 62 and 66 defined by projections 60 and 64, respectively, on the interior surface of the shell of the welding helmet (as best shown in FIG. 4). The slots 62 and 66 may be disposed proximate to the opening 58 in the shell 20.

The ADF 300 also includes a pair of engagement members 340 and 342 extending in opposing directions from one another (i.e., a first engagement member 340 extends outwardly from the first end 316 in a first direction and a second engagement member 342 extends outwardly from the second end 318 in a second direction that is opposite of the first direction). The pair of engagement members 340 and 342 are configured to be engaged with engagement mechanisms 70 and 72, respectively (see FIG. 2) disposed on the interior surface 32 of the shell 20 proximate to the opening 58 in the shell 20. When the extension members 330, 332 are disposed in the slots 62 and 66 on the interior surface 32 of the shell 20 and the engagement members 340 and 342 are engaged by the engagement mechanisms 340 and 342, the ADF 300 is removably retained against the interior surface 32 of the shell 20 proximate to the opening 58 in the shell 20 (see FIG. 4).

Referring to FIG. 5, an exploded perspective view of the various components of this example embodiment of the welding helmet 10 are illustrated. This perspective of the shell 20 shows the interior cavity 34, which is defined by interior surface 32. Side 24 includes a portion that has a through opening or aperture 40 formed therein. Proximate to opening 40 are several smaller openings 42. Opposite to opening 40 is another opening or aperture 44 formed in side 26. Opening 40 receives the outwardly extending projection 111 of mounting portion 110, and opening 44 receives the outwardly extending projection 113 of mounting portion 112. The openings 40 and 44 are sized so that the outwardly extending projections 111 and 113 can rotate therein, which allows the orientation of the headgear 100 to be adjusted relative to the shell 20. The projection 124 on the plate 122 of mounting portion 110 engages one of the group of small openings 42 formed proximate to opening 40. The engagement of projection 124 with one of the openings 42 holds the headgear 100 in place.

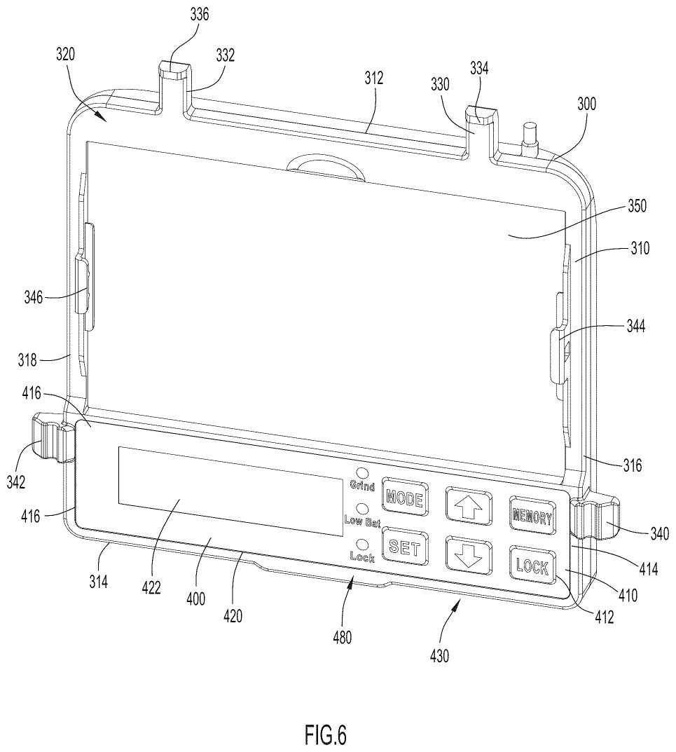

Moved away from the exterior surface 30 of the shell 20 is a shield lens 200. The shield lens 200 includes an inner surface 212, and the notches 214 and 216 extend between the inner surface 212 and the outer surface 214 (see FIG. 1). On the other side of the shell 20, the ADF 300 is moved away from the inner surface of the shell 20. The various features of this embodiment of the ADF 300 are described relative to FIGS. 6-12.

Also shown in FIG. 5 are the various features of the headgear 100. The support portions 114, 116, the forehead portion 118, and the adjustment mechanism 120, all of which can engage a user's head, are shown. The mounting portions 110 and 112 with respective couplers 130 and 132 are also shown.

Turning to FIGS. 6-12, the features of this embodiment of the ADF 300 are illustrated. Referring to FIGS. 6 and 8, ADF 300 includes a body 310 and a viewing window or viewing pane 350 that extends between the exterior side 322 (see FIG. 8) and the interior side 320 (see FIG. 6) of the ADF 300. In addition, the viewing window 350 may be centrally offset such that it is disposed within the ADF 300 more proximate to the top end 316 than to the bottom end 314. The viewing window 350 may further span across the ADF 300 between the first end 316 and the second end 318, such that the viewing window 350 is substantially rectangular. While the illustrated embodiment of the viewing window 350 is substantially rectangular, the viewing window 350 may be of any other shape. In one embodiment, the viewing window 350 of the ADF 300 may be constructed from, or include, a series of liquid crystal lens 360 and filters 362 and 364 (see FIG. 12) that may operate to alter a shade level of the viewing window 350 in order to limit or increase the amount of light passing through the viewing window 350.

Turning back to FIG. 6, the body 310 of the ADF 300 includes several components that facilitate mounting the ADF 300 to the shell 20. In this embodiment, the body 310 includes a pair of extension members 330 and 332 extending from the top end 312 of the body 310. The extension members 330 and 332 include protrusions or tabs 334 and 336, respectively, that engage the projections 60 and 64 on the interior surface 32 of the shell 20. The ADF body 310 also includes a pair of opposing lateral engagement members 340 and 342 that interact with engagement members 70 and 72, as described above. The ADF body 310 includes catches 344 and 346 located on opposite sides of the viewing window 350. The catches 344 and 346 can be used to mount an additional component, such as a lens, a screen layer, or other see through component, to the ADF body 310.

The ADF body 310 also includes a control panel 400. In this embodiment, the control panel 400 is located on the inner side of the ADF 300 and closer to the bottom end 314 than to the top end 312. The control panel 400 has a surface or face area 410 with several openings 412 formed therein. The face area 410 extends laterally or horizontally from a first side or end 414 to an opposite second side or end 416, and vertically from a top side or end 418 to an opposite bottom side or end 420. The control panel 400 also includes a setting screen 422 that generates viewable information relating to the various settings and status of the welding helmet 10.

The control panel 400 includes an array or arrangement 430 of several buttons that relate to the control of the components of the welding helmet 10. In addition, the control panel 400 includes an array or arrangement 480 of several LEDs that indicate to the user the status of the welding helmet 10. The various buttons in array 430 and the various LEDs in array 480 are described below. The location of the array 430 of buttons being closer to a side and to the bottom of the ADF body 310 makes it easier for a user to contact and press one of the buttons. In one example embodiment, the control panel screen 422 turns off or powers down after a predetermined amount of time, such as eight (8) seconds, since the last user touch of any button of the control panel 400.

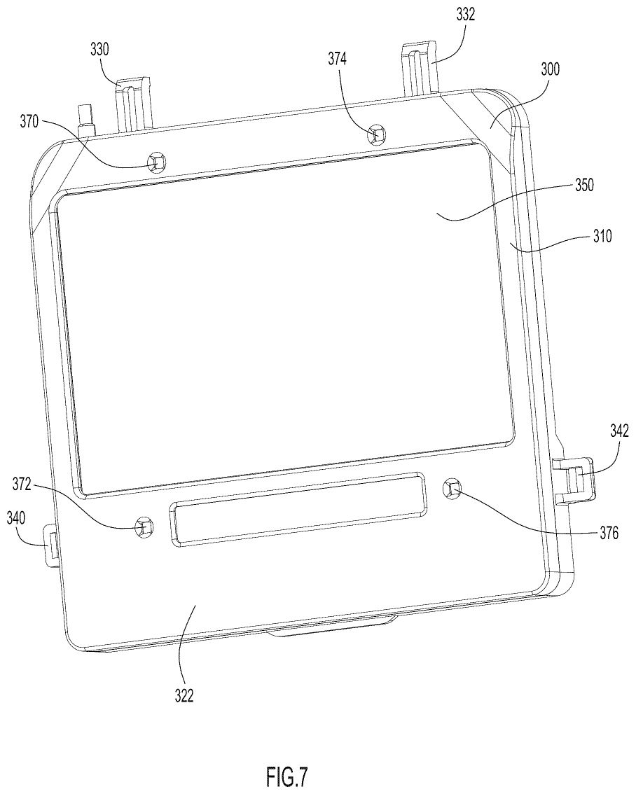

As best illustrated in FIG. 7, the ADF 300 includes a series of sensors that are disposed on the exterior side 322 of the ADF 300 around the viewing window 350. In this embodiment, the ADF 300 includes multiple sensors 370, 372, 374, and 376. The sensors 370, 372, 374, and 376 may be configured to detect the ultraviolet (“UV”) light emitted from a welding or plasma cutting arc. When one or more of the sensors detect the UV light of a welding or plasma cutting arc, the ADF 300 may be triggered to darken the viewing window 350 to a desired or appropriate shade level or setting in order to protect the welding helmet user's eyes from the harmful UV light. Returning to FIG. 3, the viewing window 350 and the sensors 370, 372, 374, and 376 of the ADF 300 are visible through the opening 58 of the shell 20 when the ADF 300 is removably coupled to the shell 20, noting that sensors 370, 372, 374, and 376 are not labeled in FIG. 3.

Also illustrated in FIG. 7 are the outer sides of the extension members 330 and 332 and of the engagement members 340 and 342. Each of the extension members 330 and 332 has a tapered or angled surface proximate to its end that facilitates the insertion of the extension members 330 and 332 into the slots 62 and 66 of projections 60 and 64, respectively.

Turning to FIG. 8, disposed below the viewing window 350 on the interior side 320 of the ADF 300 is a control panel 400. As mentioned above, the control panel 400 may include control setting screen 420 that may be disposed proximate to the first end 316 of the ADF 300 or a first end 414 of the control panel 400 (which is oriented proximate to the first end 316 of the ADF 300). The control panel 400 may further include an array 430 of buttons or actuators disposed on the opposing side of the control panel 400 such that the array 430 of buttons is disposed proximate to the second end 318 of the ADF 300 or a second end 318 of the control panel 400 (which is oriented proximate to the second end 318 of the ADF 300). As further illustrated, the control panel 400 may also include a series 480 of LEDs that are disposed between the setting screen 420 and the array 430 of buttons.

The array 430 of buttons may include a set of six buttons that are oriented in a grid-like array having two rows and three columns, with spacing or spaces 460 between adjacent rows and columns. In the illustrated embodiment, the button 440 located in the first row and first column is a “MODE” button, while the button 442 located in the second row and first column is a “SET” button. Furthermore, the button 444 located in the first row and second column is an “arrow” button pointing upwards, while the button 446 located in the second row and second column is an “arrow” button pointing downwards. As further illustrated, the button 448 located in the first row and third column is a “MEMORY” button, while the button 450 located in the second row and third column is a “LOCK” button. However, in other embodiments, these buttons may be disposed in different positions. Moreover, other embodiments of the control panel may have any number of buttons that differ from the six buttons making up the illustrated embodiment (i.e., more or less than six buttons).

With continued reference to FIG. 8, the LEDs may be aligned vertically with one another and may each emit light of a different color. In the illustrated embodiment, the top LED 490 is labeled as “Grind,” and may be illuminated when the welding helmet 10, and particularly the ADF, 300 is operating in a grind mode (e.g., the ADF 300 is adjusted to an appropriate shade level for grind operations). In some embodiments, the “Grind” LED 490 may emit a yellow color when illuminated. The middle LED 492 is labeled as “Low Bat,” and may be illuminated when the battery of the ADF 300 has a predetermined amount of battery charge left (e.g., when the battery of the ADF 300 is low on charge). In some embodiments, the “Low Bat” LED 492 emits a red color when illuminated. The lower LED 494 is labeled as “Lock,” and may be illuminated when the ADF 300 is operating in a lock shade mode where the ADF 300 will remain locked in a predetermined shade level setting regardless of the presence of any UV light. In some embodiments, the “Lock” LED 494 emits a green color when illuminated. The vertical alignment and spacing between each of the LEDs, along with the LEDs emitting different colors, enables a user of the welding helmet 10 to easily identify if one, and which one, of the LEDs is illuminated. Other embodiments of the control panel 400 may have any number of LEDs and they may illuminate any shade of color. Moreover, in other embodiments, the LEDs may be arranged in a different order and a different orientation (e.g., offset from vertical, horizontally aligned, etc.).

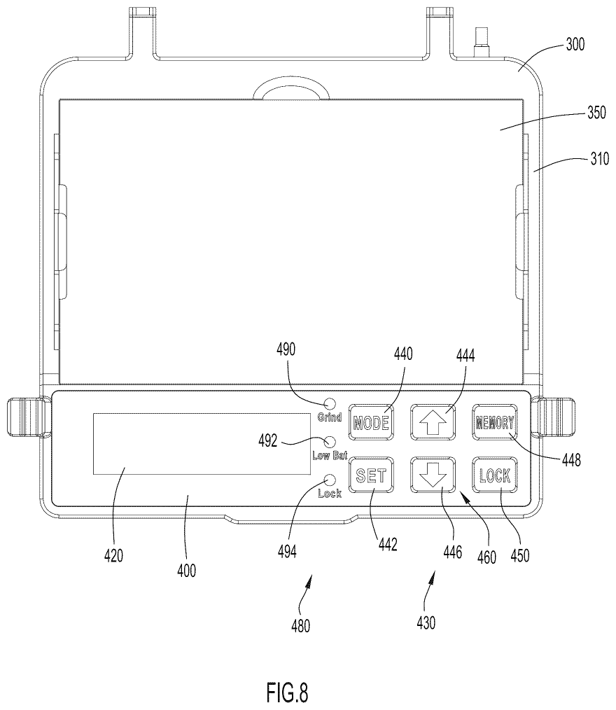

Referring to FIG. 9, the control panel 400 is illustrated by itself. The array 430 of buttons is shown in the rows and columns arrangement on the control panel 400. In particular, the MODE button 440, the upward arrow button 444, and the MEMORY button 448 are located in row 432. Similarly, the SET button 442, the downward arrow button 446, and the LOCK button 450 are located in row 434. Turning to the columns of array 430, the MODE button 440 and the SET button 442 are in column 436, the upward arrow button 444 and the downward arrow button 446 are in column 437, and the MEMORY button 448 and the LOCK button 450 are in column 438. In this arrangement, the rows 432 and 434 are oriented in a lateral or horizontal direction, and the columns 436, 437, and 438 are oriented in a longitudinal or vertical direction.

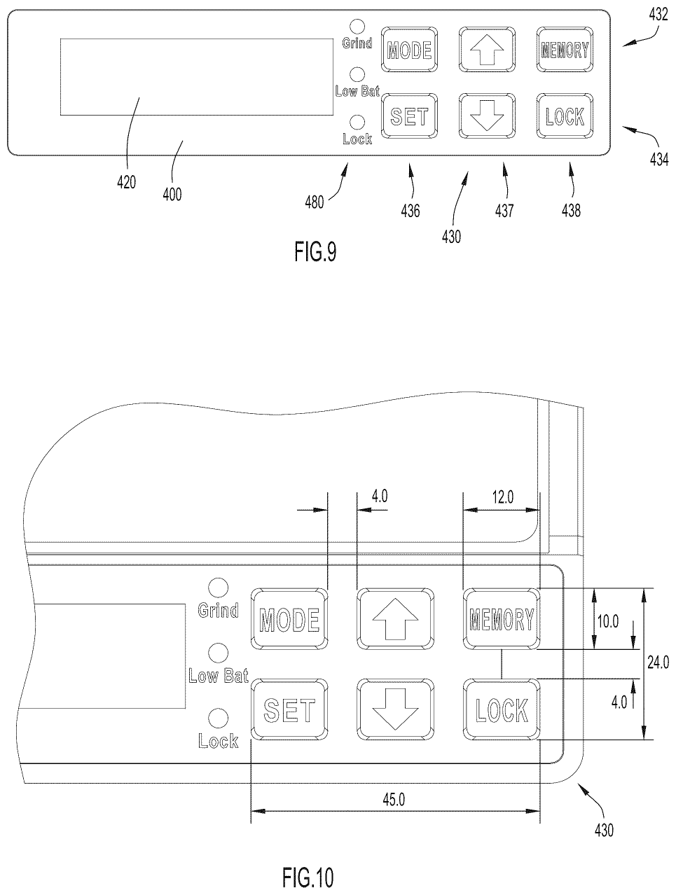

As illustrated in FIG. 10, each one of the buttons of the array 430 of buttons may be substantially rectangular having a width of at least approximately 10.0 mm and a height of at least approximately 9.0 mm, and preferably having a width of at least approximately 12.0 mm and a height of at least approximately 10.0 mm. Moreover, the buttons of the array 430 of buttons may be laterally spaced from one another (i.e., between the columns 436, 437, and 438 or in the horizontal direction that spans from the first end 316 of the ADF 300 to the second end 318 of the ADF 300) a distance greater than 3.0 mm, and preferably at least 4.0 mm. The buttons of the array 430 of buttons may also be longitudinally spaced from one another (i.e., between the rows 432 and 434 or in the vertical direction that spans from the top end 312 of the ADF 300 to the bottom end 314 of the ADF 300) a distance greater than 3.0 mm, and preferably at least 4.0 mm. Thus, the full array 430 of buttons may have a total width of approximately 45.0 mm and a total height of approximately 24.0 mm. The substantially rectangular shape of the buttons or actuators makes it easy for a user to

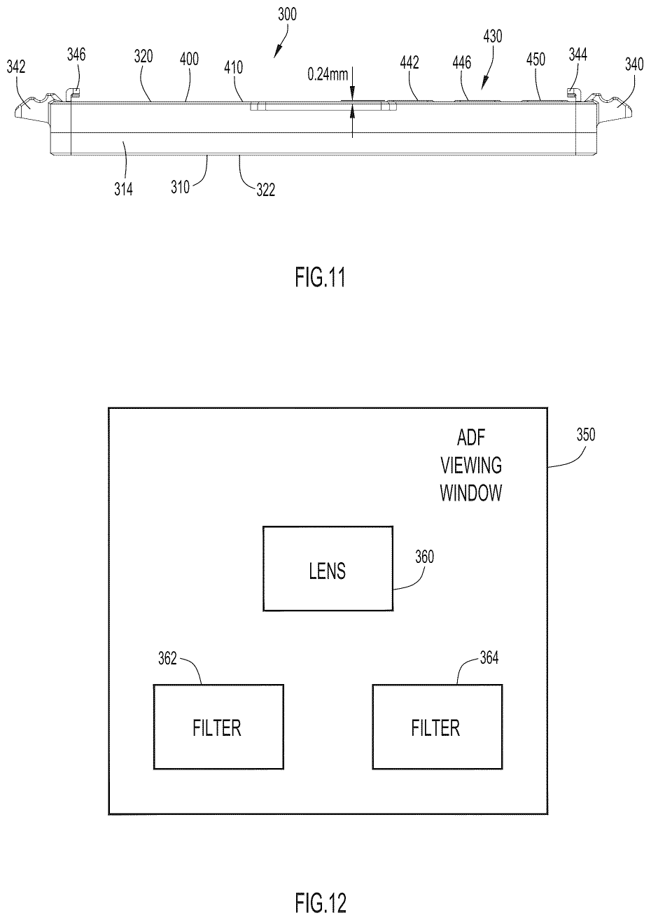

As further illustrated in FIG. 11, each of the buttons of the array 430 of buttons extends outwardly from the face or surface 410 of the control panel 400 of the ADF 300 at a distance of at least 0.20 mm, and preferably at least 0.24 mm. In this view, only buttons 442, 446, and 450 are visible extending outwardly from surface 410. The combination of the size/shape of each of the buttons, the spacing between each of the buttons, and the amount that the buttons protrude or extend from the face 410 of the control panel 400 provide each button with a tactile feel that can be felt by a user of the welding helmet 10 when wearing welding gloves. Moreover, the size/shape of each of the buttons, the spacing between each of the buttons, and the amount that the buttons protrude or extend from the face 410 of the control panel 400 also enable the buttons to be easily manipulated or actuated by a user of the welding helmet 10 while wearing welding gloves.

In FIG. 11, a bottom view directed at the bottom side 314 of the ADF body 310 is shown, with both of the opposite exterior side 322 and the interior side 320 of the body 310 being viewable. The profile of each of the engagement members 340 and 342 and each of the catches 344 and 346 can be seen in FIG. 11.

While the apparatuses presented herein have been illustrated and described in detail and with reference to specific embodiments thereof, it is nevertheless not intended to be limited to the details shown, since it will be apparent that various modifications and structural changes may be made therein without departing from the scope of the inventions and within the scope and range of equivalents of the claims. For example, the control panel presented herein may be modified to contain any number of buttons and LEDs, which may be arranged in any other arrangement, and which may have any set dimensions, provided that the arrangement and dimensions allow for a user to access a specific one of the buttons while still wearing a welding glove.

In addition, various features from one of the embodiments may be incorporated into another of the embodiments. That is, it is believed that the disclosure set forth above encompasses multiple distinct inventions with independent utility. While each of these inventions has been disclosed in a preferred form, the specific embodiments thereof as disclosed and illustrated herein are not to be considered in a limiting sense as numerous variations are possible. The subject matter of the inventions includes all novel and non-obvious combinations and subcombinations of the various elements, features, functions, and/or properties disclosed herein. Accordingly, it is appropriate that the appended claims be construed broadly and in a manner consistent with the scope of the disclosure as set forth in the following claims.

It is also to be understood that terms such as “left,” “right,” “top,” “bottom,” “front,” “rear,” “side,” “height,” “length,” “width,” “upper,” “lower,” “interior,” “exterior,” “inner,” “outer” and the like as may be used herein, merely describe points of reference and do not limit the present invention to any particular orientation or configuration. Further, the term “exemplary” is used herein to describe an example or illustration. Any embodiment described herein as exemplary is not to be construed as a preferred or advantageous embodiment, but rather as one example or illustration of a possible embodiment of the invention. Additionally, it is also to be understood that the components of the welding helmet described herein, including the control panel described herein, or portions thereof, may be fabricated from any suitable material or combination of materials, such as plastic, metals, rubbers, elastomers, natural materials, etc., as well as derivatives thereof, and combinations thereof.

Finally, when used herein, the term “comprises” and its derivations (such as “comprising”, etc.) should not be understood in an excluding sense, that is, these terms should not be interpreted as excluding the possibility that what is described and defined may include further elements, steps, etc. Similarly, where any description recites “a” or “a first” element or the equivalent thereof, such disclosure should be understood to include incorporation of one or more such elements, neither requiring nor excluding two or more such elements. Meanwhile, when used herein, the term “approximately” and terms of its family (such as “approximate”, etc.) should be understood as indicating values very near to those which accompany the aforementioned term. That is to say, a deviation within reasonable limits from an exact value should be accepted, because a skilled person in the art will understand that such a deviation from the values indicated is inevitable due to measurement inaccuracies, etc. The same applies to the terms “about,” “around,” “generally,” and “substantially.”

What is claimed is:

1. An auto darkening filter lens of a welding helmet, comprising:
a body including:
a viewing window through which a user wearing the welding helmet can view outside of the welding helmet; and
a control panel, the control panel including:
a surface defining a plurality of openings formed therein;
a plurality of actuators, at least one of the plurality of actuators being related to an operation feature of the auto darkening filter lens, and each of the plurality of actuators being located in one of the plurality of openings in the surface; and
a plurality of light emitting diodes (LEDs), wherein each of the plurality of LEDs relates to an operation of the auto darkening filter lens.

2. The auto darkening filter lens of claim 1, wherein the plurality of actuators is arranged in an array of rows and columns, and each of the plurality of actuators is spaced apart from another one of the plurality of actuators by a distance.

3. The auto darkening filter lens of claim 2, wherein the array of rows and columns includes two rows and three columns.

4. The auto darkening filter lens of claim 2, wherein the body includes a top end, a bottom end opposite to the top end, a first side, and a second side opposite to the first side, and the plurality of actuators are closer to the first side than to the second side, and are closer to the bottom end than to the top end.

5. The auto darkening filter lens of claim 4, wherein the control panel includes a screen that is closer to the second side than to the first side, and closer to the bottom end than to the top end.

6. The auto darkening filter lens of claim 1, wherein each of the plurality of actuators is a button having a width of at least 10.0 mm and a height of at least 9.0 mm.

7. The auto darkening filter lens of claim 6, wherein each of the plurality of actuators has a width of at least 12.0 mm and a height of at least 10.0 mm.

8. The auto darkening filter lens of claim 6, wherein each of the plurality of actuators is spaced apart from another one of the plurality of actuators in a horizontal direction by a distance greater than 3.0 mm, and is spaced apart from another one of the plurality of actuators in the horizontal direction by a distance greater than 3.0 mm.

9. The auto darkening filter lens of claim 8, wherein each of the plurality of actuators is spaced apart from another one of the plurality of actuators in the horizontal direction by a distance greater than 4.0 mm, and is spaced apart from another one of the plurality of actuators in the horizontal direction by a distance greater than 4.0 mm.

10. The auto darkening filter lens of claim 6, wherein each of the plurality of actuators has a substantially rectangular shape.

11. A control panel for an auto darkening filter lens of a welding helmet, comprising:
a surface defining a plurality of openings formed therein;
a plurality of actuators, at least one of the plurality of actuators being related to an operation feature of the auto darkening filter lens, and each of the plurality of actuators being located in one of the plurality of openings in the surface; and
a plurality of light emitting diodes (LEDs), wherein each of the plurality of LEDs relates to an operation of the auto darkening filter lens.

12. The control panel of claim 11, wherein the plurality of actuators is arranged in an array of rows and columns, and each of the plurality of actuators is spaced apart from another one of the plurality of actuators by a distance.

13. The control panel of claim 11, wherein the control panel includes a top end, a bottom end opposite to the top end, a first side, and a second side opposite to the first side, and the plurality of actuators are closer to the first side than to the second side.

14. The control panel of claim 13, wherein the control panel includes a screen that is closer to the second side than to the first side.

15. The control panel of claim 11, wherein each of the plurality of actuators is a substantially rectangular button having a width of at least 10.0 mm and a height of at least 9.0 mm, and each of the plurality of actuators is spaced apart from another one of the plurality of actuators in a horizontal direction by a distance greater than 3.0 mm, and is spaced apart from another one of the plurality of actuators in the horizontal direction by a distance greater than 3.0 mm.

16. A welding helmet, comprising:
a shell;
a headgear coupled to the shell, the headgear being engageable with a user's head; and
an auto darkening filter lens coupled to the shell, the auto darkening filter lens including:
a body having a viewing window and a control panel, the control panel including:
a surface defining a plurality of openings formed therein;
a plurality of actuators, at least one of the plurality of actuators being related to an operation feature of the auto darkening filter lens, each of the plurality of actuators being located in one of the plurality of openings in the surface; and
a plurality of light emitting diodes (LEDs), wherein each of the plurality of LEDs relates to an operation of the auto darkening filter lens.

17. The welding helmet of claim 16, wherein the plurality of actuators is arranged in an array of two rows and three columns, and each of the plurality of actuators is spaced apart from another one of the plurality of actuators by a distance.

18. The welding helmet of claim 17, wherein the body of the auto darkening filter lens includes a top end, a bottom end opposite to the top end, a first side, and a second side opposite to the first side, and the plurality of actuators are closer to the first side than to the second side, and are closer to the bottom end than to the top end.

19. The welding helmet of claim 18, wherein the control panel includes a screen that is closer to the second side than to the first side, and closer to the bottom end than to the top end.

20. The welding helmet of claim 19, wherein each of the plurality of actuators is a substantially rectangular button having a width of at least 10.0 mm and a height of at least 9.0 mm, and each of the plurality of actuators is spaced apart from another one of the plurality of actuators in a horizontal direction by a distance greater than 3.0 mm, and is spaced apart from another one of the plurality of actuators in the horizontal direction by a distance greater than 3.0 mm.
